(19) United States
(12) Patent Application Publication
(10) Pub. No.: US 2017/0246268 A1
(43) Pub. Date: Aug. 31, 2017

(54) Longevity Enhancement Induced by Repetitive Injections of Botulinum Toxin (Centurion Effect)

(71) Applicant: Gary E.l BORODIC, Quincy, MA (US)

(72) Inventors: Gary E. Borodic, Quincy, MA (US); Martin Acquadro, Wellesley Hills, MA (US)

(21) Appl. No.: 15/510,601

(22) PCT Filed: Sep. 14, 2015
(86) PCT No.: PCT/US15/49939

§ 371 Date: Mar. 10, 2017

Related U.S. Application Data
(60) Provisional application No. 62/049,984, filed on Sep. 12, 2014.

(51) Int. Cl. A61K 38/48; A61K 9/00

(52) U.S. Cl. CPC A61K 38/4893; C12Y 304/24069; A61K 9/0019

(57) ABSTRACT

Described herein is a novel application involving the use of therapeutic or cosmetic formulations of botulinum toxin involving an increase and enhancement of longevity in human subjects. The inventors are physicians who have used this agent for the past 30 years for many indications and has treated a large number of patients for periods of 20-30 years, many over the age of 60 at therapy initiation for medical and aesthetic conditions. A substantial number seemed to be surviving longer than untreated patients using US census statistics. Compared to social security survival curve established over the last decade, this patient group demonstrated substantial greater longevity than the national average. Because the first remarkable patients were a group exceeding or approaching 100 years of age, the phenomenon has been termed the “centurion effect.” The physiology of this effect most likely relates to the effect botulinum has on central nervous system, circadian function, sleep synchronization, mitigation of the deterioration of age related diurnal central nervous system functions, and metabolic alterations associated with the toxins effect on brainstem circadian centers.

This PCT application claims priority to U.S. Provisional Application No. 62/049,984, filed Sep. 12, 2014, which is incorporated by reference herein in its entirety.

### OVERVIEW

This patent deals with longevity, a topic that is clearly complex and multifactor in any cause-effect analysis. The inventors describes herein a lifetime of experience using one agent which appeared to relate and correlate with longevity analyzed retrospectively. The inventors understand the limitation of retrospective analysis and multiple factors that may influence cohort patient selection. However relying on the associated biologic functions such as sleep, mood and affect, light synchronization, depression, inflammation provoked senescent biologic effect, self image, multiple factors seem to intersect at an important and inspiring notion that the use of repeated botulinum application can in fact be utilized to enhance longevity. Further, the inventors have localized an effect of botulinum toxin on neurotransmitters of the brainstem conceivably related to circadian synchronization. This application target has tremendous utility in one of the most important aspects of health and welfare preservation. It is of particular note that patients analyzed and studied suffered from debilitating diseases and not cosmetic enhancement seekers. The observation made de novo by the inventors was tested against prior observations and retrospective data to derive the conclusions discussed herein.

### Aging Alters Circadian Function

Aging is typified by phase shifts and decreased amplitudes in circadian functions. These changes seem to lead to age-related circadian body temperature, melatonin secretion, sleep wake cycles, glucose and fat metabolism, locomotor activity patents and drinking behavior. The circadian wear out has been demonstrated in animals and humans with aging.

In contrast, long living mice show a high amplitude of circadian rhythms and food intake, body temperature, and a high degree “clock gene expression”. Aging is directly related to a disruption of circadian function which appears to decrease the length and quality of life. Mechanisms by which we alter circadian functions can potentially mitigate the neurologic effects that occur with aging, such as slower mentation, decreased energy, decreased physical active, and changes in body metabolic functions. Clock genes have been also shown to change with time in aging experimental animals. Knockout genetic animal models affecting certain clock genes, such as the Bmal 1 Gene, have been developed. The gene has been associated with age-related pathology such as osteoporosis, cataract, organ shrinkage, reduction in visceral and temporal adipose tissues, decreased hair growth, and chronic corneal inflammations and changes in blood cell composition against control animals. A wide variety of pathologies may indicate that certain circadian synchronization genes may be related to the senescence process. This genetic relationship indicates the import of circadian function to aging and survival.

Enhancement of a circadian function via a pharmaceutical method such as botulinum toxin can conceivably have an effect on total body function mediated by thalamic and hypothalamic activity to increase the longevity of a mammalian species. A possible mechanism is an interaction between ophthalmic mediated circadian function via an retinal thalamic neural pathway or related pathway, which improves the synchronization of central nervous system circadian functions, leading to a diminished age related effect and prolongation of life.

### Sleep Synchronization, Circadian Synchronization, and Adaption to Neurologic and Neuropsychiatric Diseases

Sleep synchronization through pharmacologic methods such as the use of botulinum toxin can in fact improve a patient's life with neurologic disease. Patients with neurologic lesions caused by blood vessel disease, multiple sclerosis, cerebral palsy, or other forms of neurologic problems can create deficits which are made worse by disordered sleep synchronization. As previously mentioned in U.S. Pat. No. 8,926,991, pain syndromes are also worsened by disruption of sleep and circadian synchronization. Botulinum toxin injections in these circumstances can improve the symptoms produced by the neurologic condition. Sleep synchronization also has been shown to be effective in patients with primary disorders of mood and affect as advocated previously by an inventor (U.S. Pat. No. 8,926,991). The selection of the use of botulinum toxin to enhance sleep synchronization can improve any form of depression, anxiety, mania, bipolar disease, and neurodegenerative diseases such as Alzheimer's. The use of this agent as a sleep synchronizer in these conditions can effectively be a mechanism by which these conditions are mitigating.

In practicing the invention described herein, in one embodiment, the use of botulinum toxin in doses 3 to 3000 LD-50 units given one or more locations in the scalp, neck, and head and neck region can effectively be useful in improving quality of life and decreasing morbidity from neurologic lesions in the brain or the peripheral nervous system such as peripheral neuropathies. Injectable formulations can be replaced by transcutaneous formulation in the form of a cream or patch if adequate deep penetration is possible. In one embodiment, an injection subcutaneously, transdermal, intramuscularly may be the preferred administration method. Nasal spray with permeation enhancers such as cationic peptide-proteins used as bulking agents to a botulinum toxin formulation. Other formulations using bipolar molecules such as lidocaine formulated with botulinum toxin can also be used. In one embodiment, the method of measuring longevity enhancement in an experimental mammalian model by virtue of injecting or applying a define amount of botulinum toxin into the periocular, face, head or neck; measuring the longevity of the mammal relative to shame controls; and performing a statistical analysis between botulinum toxin treated mammals and controls.

Sleep synchronization improvement targets certain mood and affect disorders characterized by depression as defined by the DSM IV, anxiety neurosis, or mania or any combination. Psychosocial pathologic conditions have been associated with decreased longevity, such as depression, poverty with attendant emotional and developmental impact, chronic anxiety disorders, severe post-traumatic stress and other disorders of mood and affect. Other high risk circumstances and factors can use for selection criteria to predict longevity, such as death of a parent during early childhood. In such circumstances, use of agents such as botulinum toxin may offer a physiologic advantages mitigating against a higher risk of mortality at an early age. The prophylactic use of botulinum toxin in high risk circumstances can be therapeutically useful as the side effect profile of this agent is well know when repeatedly given over many years and the agent is active on central nervous system functions involving mode and affect.

The method involves identifying a neuropsychiatric disease associated with a sleep or circadian function disorder and using botulinum toxin based on the circadian defects. As circadian dysfunction as a major selection criteria for the disorder involving mood and affect, an effective demonstration of a statistically significant response to the neuropsychiatric disorder is demonstrated against a control population. Essentially advocated herein involves 1) identifying a disorder of mood and affect and 2) identifying a disorder of sleep or circadian function and 3) using the circadian dysfunction or sleep dysfunction as a selection criterion for the treatment of the disorder of mood and affect the invention produces a favorable clinical or scientific study result. As an example, a study is designed to assess a patient population by identifying a circadian function criteria, such as a sleep disorder occurring in a patient group with a disorder of mood and affect (depression, anxiety, mania), and identifying a higher or more responsive rate of result of this circadian inclusion criteria. For instance, depression is identified as the mood affect disorder (any form including bipolar or non-reactive major unipolar depression-see DSM-4 diagnostic criteria), sleep disorder is a primary circadian criterion for inclusion into the treatment group, and using other symptoms of depression, a controlled study demonstrated efficacy of botulinum toxin to treat depression and increase longevity.

### Circadian Rhythms and Lifespan

Circadian rhythms have been previously linked to increased lifespan. Mechanisms which the circadian rhythms attenuates aging and extends life are not clearly known, however, the circadian function has been linked to an important contribution to the functioning body. In cardiomyopathic animals, experimental disruption of the circadian rhythm has been linked to a lower lifespan using light day cycle aberrations. Caloric restriction has also been linked to increase in lifespan.

As previously disclosed, botulinum toxin has been shown to have an effect on the major circadian function.

### Sleep Cycle

Sleep cycles are an important circadian function as sleep synchronization is useful in improving attentiveness, decreasing the morbidity from neurologic lesions of the brain, and maintaining a reasonable state of health. Disruption of circadian rhythms is associated with fatigue, disorientation, inattentiveness and a depressed neurologic function. The depressed neurologic function coming from the disruption of the circadian rhythm is the subject matter of the novel indication described herein.

### Circadian Rhythms in Metabolic Function in Energy Hemostasis

A most compelling linkage between metabolic disorders and the circadian clock is demonstrated in phenotypes of the clock gene mutants and knock outs. Such mutants when present in genetically altered animals have a greatly attenuated diurnal feeding rhythm. This diurnal feeding rhythm is associated with hyperphagic and obese animals with syndromes like hyperlipidemias, hepatic fatty changes, and hyperglycemia. Such alterations in metabolism have been noted to be deleterious and diabetic-like in nature. Such changes have been detrimental to longevity relative to effects on cardiovascular disease, cardiac function and peripheral arterial sclerosis. The importance of clock genes is indicating control of energy metabolism is important in overall body pathophysiology related to survival. One of the most robust phenotypic changes associated with aging is energy utilization. Both increase in body mass and increase in insulin resistance are associated with aging. Type 2 diabetes is related to body mass and insulin resistance and has long been known to be increasing in incidence in the United States and is a major contributor to coronary artery disease. Such changes that occur are clearly related to the probability of survival beyond the age of 60. The use of a circadian synchronizing agent can indeed have an effect on longevity by this mechanism.

In one embodiment, the invention is a method of altering energy utilization via facial or head and neck application of botulinum toxin through the steps of altering at least one physiologic function related to circadian cycle; alteration of energy metabolism by virtue of decreasing an age related insulin resistance; and decreasing the incidence of type 2 diabetes and subsequent increased mortality rate.

Circadian modulation of fat metabolism via cortisol and other steroid related hormones, hypertension, respiratory function (during sleep), genomic stabilization related to cancer gene expression can play an important part of the mechanism of the effect described herein. (See Savvidis et al Circadian Rhythm and cancer Biology Mol Med. 2012; 18(1): 1249-1260.

### The Suprachiasmatic Nuclei (SCN)

The circadian clock in mammals is located as previously described in the suprachiasmatic nuclei, a distinct bilateral group of cells located in the anterior hypothalamus. Destruction or alteration of the SCN leads to loss controlled bodily biologic rhythms. The master clock in the suprachiasmatic nucleus is entrained by environmental sleep light cycles. These cycles are mediated by light absorbed in the retinal photoreceptors and transmitted through retinal ganglion cells through the retinal hypothalamic track leading to the SCN. The SCN receives information on day from the retina, interprets it, and transmits it to oscillators located in the neurologic SCN via circulating hormonal factors or neuronal connections. It should be noted that the area of the SCN in high-dose administration of botulinum toxin was remarkably changed both in structure and close to toxic doses, as well as differences in an expression of choline acetyltransferase, glutamate activity and GABA activity using histochemistry on the postmortem specimens as previously described by an inventor (U.S. Pat. No. 8,926,991).

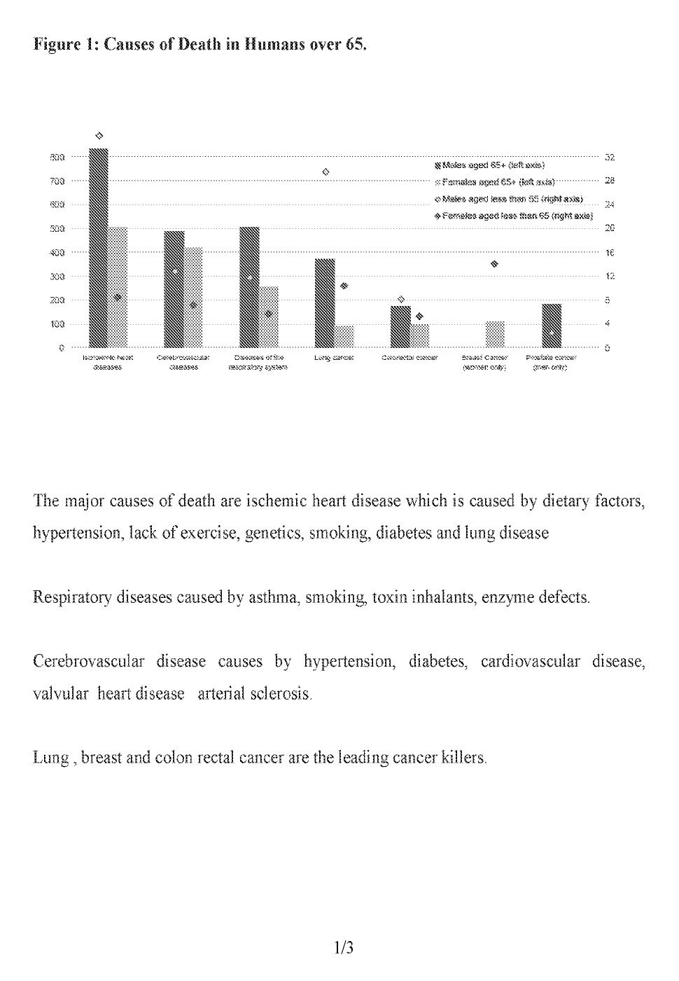

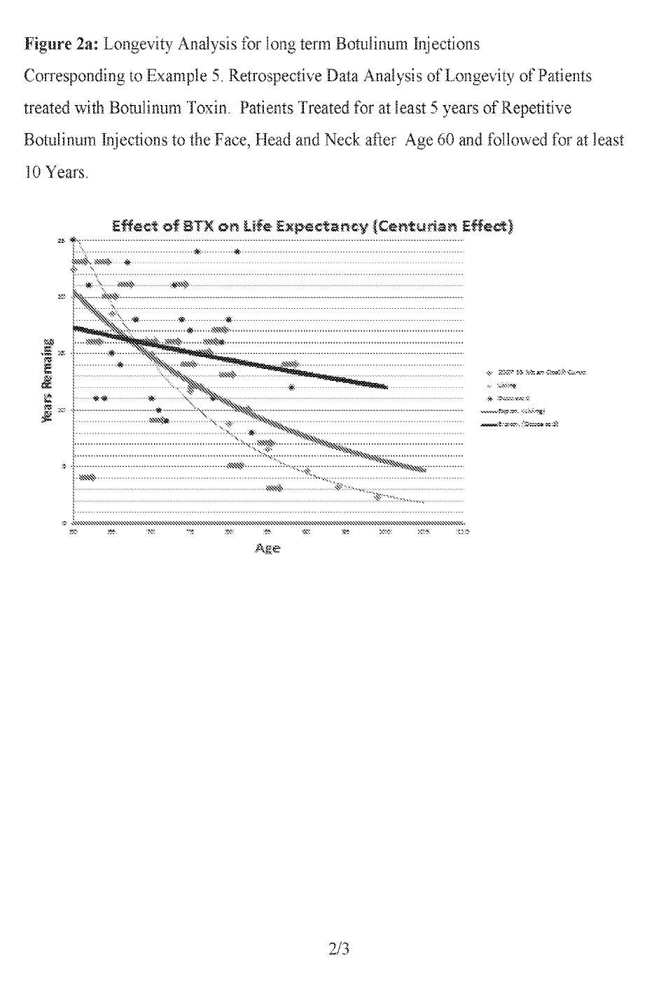

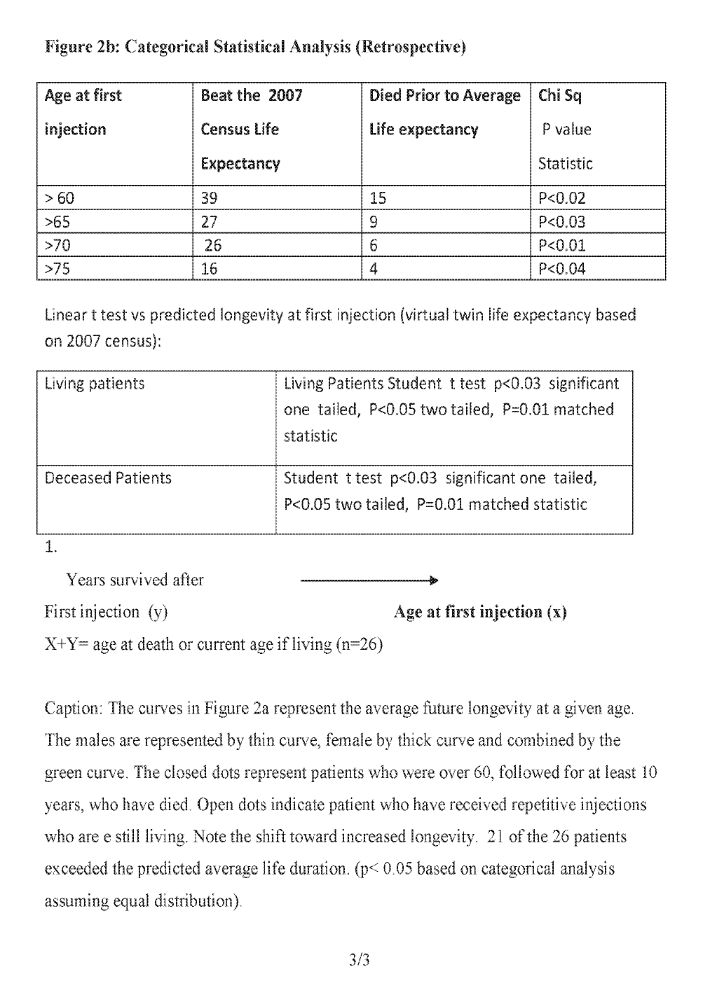

Given these findings, it is conceived in that the mechanism by which botulinum toxin injections can affect circadian rhythm and enhance the synchronization and amplitude of circadian rhythm relates to a direct effect on this area of the brain or related brain areas to the SCN. The sleep/wake cycle is the most prominent circadian function which has previously been described as affected by botulinum toxin injections. Synchronization among neurons leads to coordinated circadian outputs, regulating cellular and physiologic function throughout the body and essentially enhancing a well being and life span. Disruption of biologic rhythms has a negative effect in the short and long terms. Travelers tend to experience a jet lag which is associated with fatigue, disorientation, and insomnia. The alterations in circadian function can essentially increase the risk of hormone-related diseases, as well as a number of disorders involving disruption such as acceleration of cancer proneness and malignant growth. Malignancy and cardiovascular causes are the major causes of death in the United States of America (see FIG. 1). It should also be known that sleep synchronization has been related to blood pressure cycles. Any activity that alters the normal regulation of such functions can essentially effect duration of life.

Longevity in Hamsters with Decreased and Disruption of Circadian Rhythms

A research group has studied the effect of duration of life in hamsters with disrupted sleep synchronization. Such hamsters are noted to die at a faster rate than controls. Also in elderly hamsters receiving fetal suprachiasmatic implant seemed to restore a higher amplitude of the circadian rhythms that had diminished with age. This seemed to enhance longevity. Disruptions of circadian rhythms can lead to reduced life expectancy where appropriate resetting of circadian rhythms appears to increase the wellbeing of the animals and increase longevity.

### Eye Function and Duration of Life

Blindness has been associated with shorter duration of life. Some groups have stated there is a 50% decrease in survival time. The reasons for these observations are complex and far-reaching.

In the context of the inventions described herein, the eye is the major peripheral organ marshaling circadian synchronization. Loss of the synchronization leads to higher level of disorders of mood and affect. This leads to higher levels of sleep disorders and attendant risk of metabolic and cardiac dysfunctions related to the attendant circadian rhythm loss.

### Use of Botulinum Toxin for Mild Cognitive Impairment

The day to day work a normal individual can have a decrease in cognitive impairment, stress, anxiety, fatigue, and a feeling of overwhelming obligations can lead to cognitive impairment. The cognitive impairment can result in slowing motor function, decrease in thought process, and also in sense essential feelings of wellbeing, worthiness, and being at one's best.

Enhancement of sleep synchronization for the improvement of cognitive forms of impairment has not been wholly evaluated, however, from the observations made and the disclosures herein; cognitive improvement is a substantial effect from an enhanced sleep cycle and circadian rhythm synchronization. Cognitive improvement can occur with repetitive botulinum toxin injections and has been noted particularly in the elderly.

Age is one of the most important factors associated with reduction of cognition. It should again be noted the reduction is not pathologic and is normal with progression of life. Improvement in age related cognition by enhancing circadian synchronization, sleep-wake cycles is a beneficial target for the use of a highly safe therapy as is the case with botulinum toxin. The enhancement and preservation of cognition can be related to increase in longevity as well as essential quality of life.

In the longevity project (Terman) where 1500 children with advantaged intelligence profiles were followed for 70 years (since 1921), dramatic differences were noted in longevity comparing those with “laid back” nonproductive “comfortable,” “happy” retirements versus elderly wishing to remain productive, conscientious, contributing retirees. The latter group clearly lived longer. Although the inventors realize there may be multivariable's and confounded cause effect relationships in this analysis, this prospective observation does indicate active cognition and use of mental capacity important in longevity. H S Friedman and L R Martin in analyzing Dr. Terman's observation made an interesting reference to the Wizard of Oz:
What the Wizard said to the scarecrow, “Anybody can have a brain”. In the longevity project everybody was smart, but this is not enough to take them very far. Being intelligent was not the secret for a long life, but channeling knowledge and smarts toward productive achievement was.”

The biology of this observation is further discussed in the following section.

### Circadian Rhythm, Sleep/Wake Cycles, and Cognitive Improvement in Normal Individuals

Although botulinum toxin has been noted to be useful in neuropsychiatric disorders the application is not limited to neuropsychiatric disorders. There are many persons who have for various reasons sleep/wake cycle derangements. These are not patients with depression, major anxiety disorders, mania, or other neuropsychiatric disease. Often patients with normal day-to-day stress or who have a predisposition to desynchronization of sleep/wake cycles can have impairment and cognitive function. Furthermore, subtle changes in environment can also change the circadian rhythms adaptation of circadian rhythms such as travel, relocations, or other forms of circadian cue environmental adaption of desynchronizations.

Regardless of mechanism the invention described herein is capable of increasing the cognitive ability in a normal individual without any neuropsychiatric disorder. This means that the enhancement of amplitudes of potential circadian cycles, as well as the functional cognitive ability is enhanced. Deterioration of mental cognition is directly related to age and the derangement of mental cognition is associated with age even in patients without any evidence of neuropsychiatric problems such as Alzheimer's or any other form of neurodegeneration.

In the 1921 longevity study evaluating 1500 persons from early childhood to death (75 year study—Dr. Terman—“The longevity Project”), a continued sense of purpose and cognition was related to enhanced longevity. Cognitive enhancements such as possible with synchronized sleep cycles and circadian cycles may represent an important operational biologic system. The effect of botulinum toxin on this system provides a cognition improvement and method for cognition enhancement positively influencing longevity.

Herein describes a method of enhancement and preservation of mental cognition. Cognition means rapidity and retrieving memory, functional responses to questions or problems, in social interaction enhancements, and a motivational improvement. Also, the assessment of situational changes, work endurance, and mentation, as well as a sense of worth and recognition of issues, problems, and in day-to-day life.

Improvement in mental cognition is a substantive goal in pharmaceutical use, as long as there is the detrimental side effects of the pharmaceutical is not deleterious to other human functions. Botulinum toxin has been shown over a course of many decades to be safe as long as given in reasonable doses below defusion and dissemination values. It is repetitive of use in both cosmetic applications, as well as functional disorders of the face has proven it to have a substantial safety record making its use as an enhancement and cognition a practical and worthy application of this pharmaceutical.

### Mood and Affect, Photophobia, Extra Geniculate Photosensitivity, and Circadian Rhythm

The human retina is known to send image messages to the optic nerve from photo receptors, ganglion cells, and projections through the optic nerve to the lateral geniculate bodies where synapses are made. The synapses are made and send messages through optic radiations to the visual cortex functioning to recognize images. Additionally, a second pathway exists by which the light sensitivity through an extra geniculate pathway system to the suprachiasmatic nucleus in areas of the hypothalamus and areas of the mid brain. These extra geniculate projections govern pupillary responses. Additionally and more importantly, they govern a sensation of brightness, circadian clock rhythms, and synchronization of the circadian rhythm system. Such synchronizations are critical and most important in cue in understanding proportionment of the day cycle with intended described functions on sleep, relative to sleep, metabolic functions, cardiovascular functions, blood pressure, and other circadian functions.

Previously, the inventors have described a suppression of botulinum toxin on light sensitivity in patients with a diagnosis of Meige syndrome, essential blepharospasm, and related disorders (Borodic G E, Acquadro M A, Johnson E: Botulinum Toxin for the Treatment of Pain and Inflammation. Expert Opinion on Investigational Drugs 10(8): 1531-1544, 2001). These ophthalmologic and neurologic syndromes are often associated with hypersensitivity to light, increased blink reflex, and loss of vision through the excess of blinking and spasms. Additionally it had been noted that botulinum toxin is effective in migraine headache in which photophobia is an important component of the syndrome. The photophobia is mitigated by botulinum toxin in a vast number of patients with essential blepharopasm and Meige disease and can relieve periodic photophobia associated with migraine.

### Circadian Cycles as a Time and Age Register Monitoring Age and Survival in Population Ecology (The “Biologic Advantages” to Death and Evolutionary Genetics)

The biologic measure of circadian cycle is predominantly light cycle dependent. Taken to a more global biologic interpretation, biologic duration of life can be quantized by numbers of circadian cycles registered into brainstem centers queuing aging physiology and programmed cell deterioration. In the perspective of population genetics, the selective advantage would argue for programmed death after a certain time when the organism's reproductive potential and communal purpose no longer exists. Such programmed death would have a utility to keep the species population efficiently using availability of resources only to adults of reproductive or other forms of social biologic usefulness. Altering the light sensitivity cycle pharmacologically conceivably can delay this population driven longevity physiology, allowing a mammalian organism to survive longer periods. Stated another way, the long term biologic clock is a circadian driven function with a self destruct trigger after a critical number of cycles causing a tripping of the circadian system leading to physiologic conditions favoring dropout of older individuals necessary for preserving resources for younger individuals more useful to species preservation. This process, when viewed according to population ecology, would have a positive effect in species preservation, allowing efficient use of environmental resources. This perspective links light and circadian physiology to longevity from an ecologic perspective. Wear out of this system with repeated cycles can be linked to longevity and be a major biologic driver to age related physiologic changes. Synchronization with sleep enhancement as well as other metabolic functions such as hormonal releases, hypertension modulation, cortisol secretion plays into the concept. The de synchronization of circadian rhythm with age viewed in this perspective has biologic benefit.

Herein the inventors describe a modulation of light sensitivity seen after periocular injections which decreases the pathological light sensitivity and modulates that sensitivity so that the light sensitivity is changed and made more effective in circadian synchronization. This observation was thought to be important in the way that the toxin worked and the treatment of essential blepharospasm and Meige syndrome and have a benefit to anxiety and human depression. Circadian synchronization preservation based on botulinum toxin effects on thalamus, hypothalamus, SCN and associated brain areas can influence multiple organ systems which influences longevity.

It is notable that circadian function is generally localized to very primitive areas of the brain very universally present among mammals which has been in existence from the earliest species progenitors (paleo-encephalaon) including brainstem, a region which the inventors have previously demonstrated botulinum penetration and effect on glutamate, GABA, acetylcholine neurotransmission. If an organ was most likely to be involved in organism “self-destruction” the brainstem would be the most likely candidate based on interplay with metabolism, sleep, mood and affect, and general alertness.

Herein describes a way of modulating light sensitivity so that the circadian system is better synchronized to time of day and modulated against aberrations or desynchronization. Such desynchronization has an adverse effect on cognition, depression, anxiety, and related neuropsychiatric conditions as well as cardiovascular and metabolic functions. Attention deficit syndrome also can be enhanced by further synchronization of the circadian clock. Additionally synchronizations of circadian rhythm have been associated with increased longevity in experimental animals including hamsters and rats.

One embodiment of the invention is a method of treating attention deficit in a human without a diagnosis of a neuropsychiatric disease by enhancing sleep synchronization.

Herein the brightness synchronization produced by periocular botulinum toxin injections attendantly has such effects on circadian clock systems and therefore a potential for improvement in longevity and effectiveness of the aging mammal. The system involves regulation of inflammatory mediators, expression of peripheral clock genes, autonomic system activity, metabolic system derangements, mood and affect enhancements, cognition and alertness, decrease incidence of tumor formation, increased tolerance of non CNS tumors, inflammatory modulation, reduced the incidence of lethal events, promoting cardiovascular health, reducing hypertension, enhancing pulmonary function, altering the circadian day count and preventing programed cell death inherent within the genetic code-human genome, reducing cerebrovascular events, and fostering resistance to circadian destabilizers (shift work stress, jet lag).

### Locations and Sites of Injection

Injections are given with any formulation of type A1-5 toxin, B,C,D,E, F G toxin formulated with or without albumin, injected into facial regions or head and neck regions, at intervals between 1-52 weeks or applied through a transcutaneous, trans nasal otic canal, Aerosol, oral formulation, zinc enhanced formulation, chimeric associated protein for penetration or localization. Bulking agents such as polycationic proteins, poly lysine, or other nerve or epithelial penetrating agents can be used. Most preferably the injection are given in forehead, periocular region, scalp in doses of 10-500 U in multiple locations at 4-16 week intervals repetitively. These locations enhance central nervous system effects on brain areas governing circadian function.

### Preferred Formulation and Botulinum Properties

Given the botulinum toxin penetration into the central nervous system would be the most likely mechanism; the invention herein is not limited to a specific mechanism but an observation of a specific utility. However, given the most likely mechanism involves a central nervous system effect formulation which offer this creates permeation factors and uptake by neurons would be preferable. Such formulation would include the most potent version of botulinum toxins A1-A5 (A1, A2 most preferable). Further permeation and uptake agents such as proteins, protein enzymes, lipids, or polysaccharides which enhance permeation into the Central nervous system would potentially enhance the desired effect. Use of bulking and permeation or neuron uptake agents devoid of human blood products is preferable. The goal of the added excipients is to stabilize the active neurotoxin for portability, enhance penetration through the blood brain barrier, and enhance neuronal cell uptake within the peripheral and central nervous system.

Circadian Function. Inflammatory Diseases, and Pulmonary Function

Circadian disorders have been associated with increased morbidity of certain inflammatory conditions such as inflammatory bowel disease, Rheumatoid arthritis, asthma, eczema and other related conditions. Such interplay between circadian rhythm and critical pulmonary disease can be related independent of effects on mood and affect or as a consequence of effects on anxiety depression and sleep. By virtue of enhancing the circadian rhythm, positively influencing sleep habits, and enhancing other elements of circadian system, respiratory pathology can be mitigated.

### Circadian Function and Breast Cancer

Circadian disruption has been linked to progression of breast cancer based on assessment of biomarkers (Cash E et al. Circadian disruption and biomarkers of tumor progression in breast cancer patients awaiting surgery. Brain Behav Immun 48:102-14 2015). Loss of circadian clock gene expression has been associated with progression of breast cancer (Cadenas C et al Loss of Circadian clock gene expression is associated with tumor progression in breast cancer Cell Cycle 13:3282-91,2014. Circadian disruption has been associated with resistance to hormonal therapy (Dauchy R T et al. Circadian and melatonin disruption by exposure to light at night drives intrinsic resistance to tamoxifen therapy in breast cancer. Cancer Res 74(15) 2014 Night shift work has been associated with an increased risk of breast cancer (Endocr Relat Cancer 21:629-38,2014). Alternating light cycle has been associated with increased risk of breast cancer among mice.

From the above it is clear that positively altering a regular sleep and associated circadian function can alter and interact with longevity both from an incidence of breast cancer but also with respect to tumor progression and response to therapy. As breast cancer is a leading cause of death among woman, attention to expression of clock genes, circadian interactions, and circadian rhythm are important in therapeutic approaches and tolerance in this malignancy. Botulinum toxin effect on synchronization and enhancement of the circadian cycle can positively benefit this common malignant condition.

### Circadian Function and Prostate Cancer

One epidemiologic study has examined sleep duration in relation to prostate cancer risk. In a cohort of Japanese men, sleep duration was inversely associated with risk of prostate cancer). Compared with those who slept an average number of hours (7-8 hours), those sleep deprived (6 hours or less) were at increased risk of developing prostate cancer, whereas those who slept for longer than average (9 or more hours) were at lower risk for prostate cancer. The association between short sleep duration and prostate cancer risk was stronger for advanced disease defined as prostate cancer stage T3/T4 and/or metastasized. The inverse association of sleep duration and prostate cancer risk in this study is in line with observed increased nocturnal melatonin secretion with longer sleep duration and decreased melatonin levels in prostate cancer patients.

Prostate cancer is a leading cause of cancer death among men and its incidence can conceivably be altered and reduced by increased circadian synchronization.

### Circadian Rhythm and Cancer

In 2007, the International Agency for Research on Cancer of the World Health Organization designated shift work involving circadian disruption as “probably carcinogenic to humans”. The main rationale for this classification is evidence from experimental animal models and limited evidence from human epidemiologic studies describing an increased risk of breast cancer among long-term female night shift workers, including flight attendants, as compared with women who do not work during the night.

### Circadian Clock Genes in Peripheral Tissues and Cancer

A number of clock genes in peripheral tissue are involved in cell cycle regulation. Recently, circadian gene TIMELESS is expressed in cells of advanced breast cancer.

This gene is thought to regulate the peripheral organ circadian cycle with that of the central nervous system. Botulinum toxin activity both in the periphery acting on gene regulation and within the central nervous system can modulate and therapeutically benefit those afflicting with malignancy in which clock gene disruption in peripheral tissue and central nervous system are involve with tumor incidence and progression.

Peripheral tissue injections can be given according to a diffusion nomogram previously described by the inventors for the purpose of targeting peripheral tissue clock genetics to mitigate malignant cell transformation. The interaction of botulinum toxin both at the brainstem level and at the peripheral nervous system and peripheral organ tissues to alter diurnal variations of gene expression, metabolic cell cycles can have an effect on tumor incidence and behavior neoplastic cell transformation once cancer has occurred.

Here the application of botulinum toxin is given to the face, scalp, head or neck, periocular area to effect a positive clinical out in the remote organ by altering the circadian cycle in that organ. The remote organ benefits from the altered cycle to improve the chance of malignant cell transformation, and damage produced by the malignant cells one transformed.

In one embodiment, the method taught herein A method of decreasing age related cancers by facial or head and neck application of botulinum toxin, thereby altering at least one physiologic function related to circadian cycle.

### Circadian Rhythm and Cardiovascular Disease

Myocardial infarction (heart attack) and ventricular arrhythmia (sudden death causes) exhibit a circadian rhythm relative to incidence. Both of these conditions comprise cardiovascular event often leading to death. Diurnal variation occurs in autonomic nervous activity, plasma cortisol, renin-angiotensin activity. Clock gene desynchronization between the central nervous system and peripheral clock gene structure may promote inflammatory reactivity in the peripheral vessels and tissues.

Endothelial function and platelet function, blood pressure, and heart rate undergo diurnal variations. Barorefelx sensitivity (controlling blood pressure) is under diurnal variation. Night shift work and jet lag are risk factors toward myocardial infarction, the leading cause of death in the human race. Response to blood pressure medication has been noted by various authors to be diurnally dependent. Remodeling of heart tissue after myocardial infarction has been noted to be diurnally dependent.

Here the application of botulinum toxin is given to the face, scalp, head or neck, periocular area to effect a positive clinical out in the remote organ by altering the circadian cycle in that organ. The remote organ benefits from the altered cycle to improve the inflammatory response and mitigate the damage produced by the cardiovascular event.

Further appetite and food consumption with attendant metabolism is under diurnal control. The metabolic syndrome (diabetes, hyper-lipidemia, central accumulation of adipose tissue) is often associated with vascular disease. The circadian cycle regulates appetite and desynchronization has been linked to alterations of energy-tissue accumulations. It is well know that clock genes exist on peripheral tissues inclusive of blood vessel endothelium. Alteration of circadian signal in the periphery can negatively influence blood vessel heath leading to accelerated arterial sclerosis. Impairment of the peripheral circadian tissues genes has been associated with increased inflammatory pathology leading to vessel closure and pathology.

### Circadian Rhythms and Cerebrovascular Disease

Repeated studies have shown an increased incidence of stroke in early morning. This effect mimics the incidence of heart attacks during this time period. Strokes can be ischemic, hemorrhagic or embolic. The ischemic stroke was the most closely linked to circadian rhythm. Also cerebral small vessel disease has been linked to disturbed 24 hour activity rhythms. Sleep disturbances such as primary insomnia as well as sleep apnea have also been lined with an increased incidence of stroke.

Recently, a large study (Wu M P et al. Apr. 14, 2014) have demonstrated primary insomniac have a significantly increased incidence of stroke, particularly among younger patients. They further demonstrated the incidence correlated roughly with the degree of insomnia. Insomnia here can provoke undesirable inflammatory changes and immune system changes leading to increased risk.

As stroke represents the second largest cause of death, any therapy which enhances synchronization of sleep such as botulinum toxin can further have reaching benefits to prophylaxis in this disease category.

Here the application of botulinum toxin is given to the face, scalp, head or neck, periocular area to effect a positive effect altering the circadian cycle such as sleep and attendant physiologic consequences therapy synchronizing sleep and other mentioned circadian function causing a decreased incidence of stroke, or a decreased recurrence of stroke.

### Rheumatoid Arthritis

In the incidence of rheumatoid arthritis ha been demonstrated to be higher among women who are late shift workers. Among the symptoms of patients with rheumatoid arthritis (RA), joint stiffness is influenced by diurnal rhythm and reaches peak in the morning, which is a common complaint and reflects the circadian nature of disease manifestation. In addition, inflammatory cytokines, which reach peak secretion early in the morning are major players causing the morning stiffness. The link between the circadian clock and inflammation, focusing on the interactions of various clock genes with the immune-pathways underlying the pathology of rheumatoid arthritis.

People with rheumatoid arthritis (RA) who don't sleep well face significant risks of greater functional disability due to pain and fatigue symptoms associated with poor sleep quality, a new study shows.

Researchers at the University of Pittsburgh studied the relationship between sleep quality and functional disability in 162 patients with rheumatoid arthritis. All patients had been diagnosed with RA for at least two years; their average age was 58.5 and 76% were female.

The primary finding of our study is that poor sleep quality is associated with greater functional disability among patients with [rheumatoid arthritis] and this relationship may be explained by pain severity and fatigue

Here the application of botulinum toxin is given to the face, scalp, head or neck, periocular area to effect a positive effect altering the circadian cycle such as sleep and attendant physiologic consequences therapy synchronizing sleep and other mentioned circadian function causing decrease in inflammation, symptoms and disability associated with rheumatoid arthritis.

### Asthma and COPD (Chronic Obstructive Pulmonary Disease)

People with asthma and/or COPD who have a frequent problem waking up at night often have worse respiratory disease. They are also at risk for complications from their asthma or COPD including worsening of disease progression from inflammatory mechanisms. Sleep disturbances can worsen asthma symptoms throughout the day, increase your need for rescue inhalers, and worsen quality of life. Synchronizing the circadian rhythm using botulinum toxin can mitigate the progression and morbidity associated with these diseases.

Here the application of botulinum toxin is given to the face, scalp, head or neck, periocular area to effect a positive effect altering the circadian cycle such as sleep and attendant physiologic consequences therapy synchronizing sleep and other mentioned circadian function causing a decreased incidence of stroke, or a decreased recurrence of stroke.

Here the application of botulinum toxin is given to the face, scalp, head or neck, periocular area to effect a positive effect altering the circadian cycle such as sleep and attendant physiologic consequences therapy synchronizing sleep and other mentioned circadian function causing decrease in inflammation and symptoms of asthma, COPD and mitigation against progression of disease.

### Invention Not Limited by Mechanism:

Described herein is a method for increasing the chances for longevity using repetitive botulinum toxin and should not be limited by any theory. The existing relationships are given for purpose which could relate plausibility to the observation and would be subject to future study however the consistency of the observation is unexpected and serendipitous.

### Definitions Described Herein.

Circadian rhythm disorder as described herein refers to non 24 syndrome, sleep disorders, awake sleep cycle disorders, disorders of biologic rhythms in a fixed controlled environment, melatonin related disorders involving biologic rhythms, cyclic appetite related de synchronization, hypothalamic endocrine fluctuation disorder, cortisol fluctuation disorder.

Longevity refers to duration of life.

Botulinum toxin refers to any immunotypes or sub immunotypes.

Human subject refers to any person allowing treatment to obtain a benefit.

Controlled animals study refers to an animal study which shame applications and measures duration of life.

Injection means application of agent at any depth through animal tissues.

Statistically significant means a comparison which has a probability of there being a difference at 95%

### EXAMPLES

### Example 1

A 103 year old woman received repetitive botulinum injection for about 25 years. She notes repeat decrease in light sensitivity and has maintained a mobile, lucid cognition until this day. Botulinum injections were repeatedly given from age 85 forward at doses between 40-100 units every 6-12 weeks. She maintains a good short term memory, sleeps regularly and processes questions quite normally. There was no history of cardiovascular disease or hypertension. This patient was noted to be less anxious and sleeps better after injections.

### Example 2

A 100 year old man received repeated botulinum injections for about 5 years for blepharospasm. Doses ranged from 40-80 U. He lived until 100 years old. During all follow-up visits, he was noted to maintain a good memory, ask insightful questions, and relate appreciate concerns regarding his medical condition.

### Example 3

A 95 year old man received about 10 years of repeat botulinum toxin for over 10 years started at age 78. He is 95 living a reasonable existence. He has been able to actively work until almost 90 years old and support his daughter's aspirations to attend medical school after her 50^{th} birthday. His sleep pattern was noted to be normal. This patient had no hypertension.

### Example 4

A group of patient noted to be greater than 60 years old who received botulinum toxin injections repetitively for at least 5 years were retrospectively analyzed for duration of life compared to the 2007 Social security life expectation curve. Each patient needed at least 10 year follow up from initial injection. The majority of patient both living and deceased exceeded the life expectancy predicted by the census curve published in 2007. This was noted to be true both of patients deceased and those still living.

The shift of life expectancy over the average was unexpected and a novel observation, serendipitous outcome and utility of the benefits of botulinum toxin in this patient subset.

### Example 5: (Retrospective Population Study)

Inventor retrospectively analyzed patient from his practice who were treated for at least 5 years with repetitive botulinum toxin injections ranging from 20-300 units per cycle and older than 60 years at the time of botulinum injection initiation. The median injection was about 50-70 units per cycle. In some injections were given in the head and neck with doses ranging from 20-400 units. The group of patients were diagnosed generally with movement diseases such as Meige syndrome (cranial facial dystonia), essential blepharospasm, hemifacial spasm, dystonia, bruxism). Each record was originated after the year 1982 and represented a cohort of patients treated for various other conditions. Each of these conditions involved injections on the face, head, or neck. More commonly injections were given in the periocular regions in one or more points (usually 4-20 points but rarely as many as 40 points). Deceased patient were included in the study as well as living. Date of death was obtained from patient record and/or internet obituary postings. No normalization was made for co existing malignant conditions. The early analysis of data is given in FIG. 1 comparing longevity to the statistical average. Note that the majority of patient were noted to live longer at the curve, both living and dead. The arrows depict patient still alive whereas the black dots indicate the deceased. A statistical analysis comparing the patient at the time of induction to a virtual person with longevity predicted by the 2007 census and categorical comparisons conducted using standard 1 tailed chi square. Table 1 compared the patient with a virtual longevity person (census based). Note that each bin shows a statistically significant for each grouped 5 year bin between the age of 60-65, 65-70, 70-75, 75-80, 80-85. The data in a compilation of about 61 patents. Note that as many patients are still living, the categorical comparisons will become even more significant if updated within the next several years. Further that many of the deceased occurred in prior decades than current when earlier censuses survival was reduced compared to 2007. It is noted that the average survival is increasing in the United States however the latest consensus was the reference. The inventors acknowledge there are many confounding issues using retrospective data and biases relative to cohort selection. Such biases include but not limited to committed patients, socioeconomic factors, racial factors and genetic factors, and possible genetic linkages of longevity genetic polymorphisms linking the various diseases to longevity as well as other factors. The intuition of the inventors is that the [symbol] retrospective data is sufficient to formulate a targeted purposeful indication on the use of botulinum toxin as a method extending survival irrespective of mechanism.

### Example 6: Use of Mammals and Demonstrating the Centurion Effect Against Shame Controls

Here, animals are enormously useful to prospectively study the effect of botulinum toxin on longevity. Animal studies have been highly effective in linking reduced food consumption with longevity as well as a number of other factors.

Here an animal population with known statistical longevity could be divided into two groups. Control cohort receiving placebo and study cohort receiving repeated non lethal injections of botulinum toxin at fixed or variable intervals. The study endpoint is death and the life duration can be analyzed under a linear or categorical statistical method. Shame or controls are used for statistical comparison using categorical or other forms of statistical analysis using probability tables (p values using a 95% or greater confidence level as a significant difference).

Additionally stresses to longevity can be applied to each study group such as a select transgenic rodent with knockout genes to circadian clock proteins regulating sleep, environmental stresses such as excess feeding, light de synchronization, or any other environmental factor causing reduced longevity. Such risk factor added environmentally or via genetic selection can be used test the effect of longevity enhancement described herein.

Further aged animals can be tested. Here it would be important to select for animals with no obvious sickness and those with no biases (selection criteria) to being inducted for study.

The expected and predicted result is the botulinum injections become a useful factor in life span preservation and enhancement.

The use of animal studies helps control against testing the notion in a prospective format controlling against biases inherent in any human study.

### Example 7: Neuronal Cell Cultures

The pharmacology of the effect can be studied using neuronal cell cultures. Neurons can be harvested from animal spinal cords or brain tissues so that the effect on cell integrity and function over time can be studied. The purpose of the studies objective would be to focus on longevity and versatility of the cells to resist stress in the environment or external stress associated with cell death.

In one embodiment, the method of measuring longevity enhancement in neuronal cell cultures by virtue of injecting or applying a define amount of botulinum toxin into the cell culture; measuring the longevity of the neuronal cells to sham controls; and performing a statistical analysis between botulinum toxin treated neurons and controls.

It is anticipated that alternate cell systems can be used to study this effect.

### Example 8: Improving CNS Penetration

A botulinum toxin with excipients useful for biologic membrane barrier penetration is employed to produce an enhancement in longevity. Such enhancements can include certain protein additive rich in ionic amino acid side chains, high concentration protein carriers such as albumin, poly lysine, lidocaine and associated anesthetics. The volume of the delivery injection may be increased to allow enhance central nervous system delivery.

### Example 9: Prophylactic Administration Circadian Function and Longevity

A patient is treated with botulinum toxin over the face, forehead head and neck and is noted to be more alert, have a more synchronized and regular sleep, appetite, blood pressure, glucose metabolism, cortisol levels, body temperature, gastric motility and gastric hormonal function, and a lower incidence of lung, breast, and prostate cancer. The administration decreases the incidence of type 2 diabetes by synchronizing sleep, appetite and temperature with attendant changes in metabolism.

### Example 10

A patient is known to have a strong family history of cardiovascular disease. Botulinum toxin is given to the face, head and neck, or periocular area by injection or topical application thereby reducing the risk of experiencing a heart attack or a lethal arrhythmia such a ventricular tachycardia leading to ventricular fibrillation. Longevity is preserved.

### Example 11

A patient is known to have a strong family history of breast cancer or has a genetic makeup predisposing to breast cancer (e.g., BRCA gene positive). Botulinum toxin is given to the face, head and neck, or periocular area by injection or topical application thereby reducing the risk of experiencing breast cancer or the lethal ramifications such as metastasis leading to lung, bone and brain involvement leading to death. Longevity is preserved

### Example 12

A patient is known to have either a strong family history or multiple risk factors for cardiovascular disease. Botulinum toxin is given to the face, head and neck, or periocular area by injection or topical application thereby reducing the risk of experiencing a heart attack or a lethal arrhythmia such a ventricular tachycardia leading to ventricular fibrillation. Longevity is preserved

### Example 13

A patient is known to have prostate cancer. Botulinum toxin is given to the face, head and neck, or periocular area by injection or topical application thereby reducing the risk of experiencing lethal ramifications such as metastasis leading to lung, bone and brain involvement leading to death. Longevity is preserved

### Example 14

A patient is known to have an increased risk of stroke based on age, other forms of cardiovascular or peripheral vascular disease, hypertension, strong family history, or previous stroke

Botulinum toxin is used to improve insomnia, sleep patterns, and circadian function synchronization thereby reducing the risk of cerebrovascular disease.

Botulinum toxin is given to the face, head and neck, or periocular area by injection or topical application thereby reducing the risk of experiencing lethal ramifications of stroke such as paralysis, impaired cognition, speech loss (aphasia, dysarthria), coma, disturbed central breathing, blindness, coordination, disorientation, seizures and death. Longevity is preserved

### Examples 15

A patient is known to have a rheumatoid arthritis or is a high risk (shift worker, family history, and biologic markers-autoantibodies).

Botulinum toxin is used to improve insomnia, sleep patterns, and circadian function synchronization thereby reducing the risk of progression of rheumatoid arthritis and decrease symptoms of the disease.

Botulinum toxin is given to the face, head and neck, or periocular area by injection or topical application thereby reducing the risk of experiencing lethal ramifications such as heart failure, increased infections. Longevity is preserved

### Example 16

A patient with asthma or COPD is identified with a sleep disorder or circadian defect. Botulinum toxin is used to improve insomnia, sleep patterns, and circadian function synchronization thereby reducing the risk of progression of asthma or COPD. Botulinum toxin is given to the face, head and neck, or perio ocular area by injection or topical application thereby reducing the risk of experiencing lethal ramifications of COPD or asthma such as respiratory crisis, pulmonary scarring, fibrotic lung disease, emphysema, and acidosis. Longevity is preserved

1. A method of statistically increasing the chances of longevity in human by the administration of a botulinum toxin
a. selecting a patient; and
b. administering the botulinum toxin to the patient;
thereby increasing longevity in the patient.

2. The method of claim 1, wherein the botulinum toxin includes botulinum type A1-A5, B,C,D,E,F or G.

3. The method of claim 1, wherein the botulinum toxin is is injected into the face, head or neck.

4. The method of claim 1, wherein the botulinum toxin which involves the application of cream, ointment or lotion to head and neck or face or periocular region.

5. The method of claim 1, in which the patient is injected with between 1-3000 LD 50 units of botulinum toxin type A.

6. The method of claim 1, in which the patient is injected with between 1-40,000 units of botulinum toxin type B.

7. The method of claim 1, in which botulinum toxin is administered using aerosols to nose lungs, otic canals.

8. The method of claim 1, in which botulinum toxin is administered through repeated injections.

9. The method of claim 8, in which the repeated injections are administered at intervals between 2-52 weeks.

10. The method of claim 1, in which the selected patient is a human over 60 years old.

11. The method of claim 1, which causes at least one change in circadian function.

12. The method of claim 11, wherein the change in circadian function includes metabolic alteration in fat, sugar, or protein metabolism, and altered inflammatory response.

13. The method of claim 11, wherein the change in circadian function involves alteration in blood pressure, appetite, energy level, cognitive ability, and alertness.

14. The method of claim 12, wherein cognitive ability is increased without a diagnosis of a neuropsychiatric disease by enhancing sleep synchronization.

15. A method of decreasing the incidence of cardiovascular disease by
a. administering botulinum toxin to the face or neck; and
b. altering at least one physiologic function related to the circadian cycle.

16. The method of claim 15, in which a change in circadian cycle modulates and reduces hypertension.

17. A method of preserving normal cognition in aging human without a diagnosis of neuropsychiatric disease by
a. administering botulinum toxin to the face or neck; and
b. altering at least one physiologic function related to the circadian cycle.

18. The method of claim 17, in which the alteration of circadian function is increased sleep synchronization.
